(19) United States
(12) Patent Application Publication
(10) Pub. No.: US 2025/0134736 A1
(43) Pub. Date: May 1, 2025

(54) DIAGNOSTIC ARRANGEMENT FOR SANITARY NAPKIN TO DETECT AND ANALYZE ANALYTES IN MENSTRUAL BLOOD

(71) Applicant: PES UNIVERSITY, Bengaluru (IN)

(72) Inventors: Meghna Ravishankar Valakatte, Bengaluru (IN); Sowmyaa Shridhar, Bengaluru (IN)

(21) Appl. No.: 18/401,744

(22) Filed: Jan. 2, 2024

(30) Foreign Application Priority Data
Oct. 31, 2023 (IN) 202341074277

(51) Int. Cl. A61F 13/84; A61B 5/15; G16H 40/67

(52) U.S. Cl. CPC A61F 13/84; A61B 5/150045; G16H 40/67; A61F 2013/8491

(57) ABSTRACT

The present disclosure teaches a sanitary napkin having diagnostic arrangement to detect analytes in menstrual blood. The sanitary napkin includes a top layer to collect the menstrual blood and a middle layer having vertical columns for size-based separation of the collected menstrual fluid. Further, the sanitary napkin includes a sensor layer having spots corresponding to the vertical columns to sense concentration of the analytes. Further, the sanitary napkin includes a transmitter to transmit the sensed concentration to a user device for comparing with healthy conditions to detect and analyze the analytes in the menstrual blood. Additionally, the sanitary napkin includes a bottom layer to sandwich the middle layer, the sensor layer, and the transmitter with the top layer.

### FIELD OF THE PRESENT DISCLOSURE

Embodiments of the present invention generally relate to the field of female health and hygiene. In particular, embodiments of the present invention relate to diagnostic arrangement to detect and analyze analytes in menstrual blood.

### BACKGROUND OF THE DISCLOSURE

Vaginal infections are typically caused by yeasts and bacteria which tend to overgrow in the presence of high estrogen. Further, since the level of estrogen during the menstrual cycle is high, the risk of such vaginal infections is particularly high around the time of menstruation. During such vaginal infections, the major causative organisms are facultative anaerobic bacteria like Staphylococcus aureus and Escherichia coli, and fungi in the vaginal microbiome like Candida albicans. However, such vaginal infections may usually go undetected and get resolved automatically, but if they are not resolved and detected on time then such vaginal information may lead to life-threatening effects. While the awareness on menstrual health and hygiene has increasingly been advocated, nevertheless there also stands a pressing need to develop rapid and accurate point-of-care methods to diagnose at an individual level.

Conventionally, in order to detect such vaginal infections, a user has to get a pelvic examination done for collecting a sample of vaginal fluid through swabs/smears that are sent to laboratories for confirmation through molecular tests and biochemical assays. This is highly inconvenient to the user due to many reasons, such as privacy, uncomfortableness, invasiveness, and time consumption. In order to overcome such issues, at-home kits are used that usually involve pH-based testing which may be less invasive and uncomfortable for the users. However, some labs may provide facilities to collect samples at home followed by a detailed laboratory diagnosis, nevertheless, such tests still remain time-consuming, invasive, and bring in a factor of discomfort.

Thus, there is a need for a vaginal infection-detecting mechanism that overcomes the above-mentioned drawbacks of the conventional solutions and which is non-invasive, reliable, faster, and convenient for the user.

### BRIEF SUMMARY OF THE DISCLOSURE

An embodiment of the present disclosure discloses a diagnostic arrangement to detect analytes in menstrual blood. In an embodiment, the diagnostic arrangement includes a top layer to collect the menstrual blood. The diagnostic arrangement further includes a middle layer having a plurality of parallelly placed vertical columns for a size-based separation of the collected menstrual fluid. The parallelly placed vertical columns include one or more shapes with one or more detection markers coated on a corresponding inner surface to react with one or more analytes in the collected menstrual fluid. The one or more shapes include a funnel shape, a rolling pin shape, a conical tapering shape, and/or a cylindrical shape. In an embodiment, the one or more detection markers include antibodies specific to antigens of interest and oligonucleotides bound to complementary nucleic acids. Further, the size-based separation forms a first region of larger-size components and a second region of smaller-size components. The larger size components include cellular matter having endometrial cells, White Blood Cells (WBCs), and Red Blood Cells (RBCs), and the smaller size components include bacteria along with plasma content having proteins and nucleic acid.

In an embodiment, the diagnostic arrangement includes a sensor layer having a plurality of spots corresponding to the plurality of parallelly placed vertical columns, such that each spot of the plurality of spots senses the concentration of one of the one or more analytes across the corresponding vertical columns. In some embodiments, the sensor layer has impedance-based sensors on each of the plurality of spots, such that the coupling of an antigen with the detection marker leads to a change in impedance on the corresponding spot. Further, the changes in impedance on the corresponding spot are associated with a concentration of a corresponding analyte binding in the corresponding vertical columns. The analytes facilitate the detection of infectious disease conditions including Candidiasis based on fungi Candida albicans, Gonorrhea caused by Neisseria Gonorrhea, and menstrual-associated Toxic-Shock Syndrome (mTSS) caused by Staphylococcus aureus.

In an embodiment, the diagnostic arrangement includes a transmitter to wirelessly transmit the sensed concentration to a user device to compare with known normal healthy conditions for detecting and analyzing the one or more analytes in the menstrual blood. In an embodiment, the top layer, the middle layer, the sensor layer, and the transmitter are included in a sanitary napkin, a tampon, and a detection strip.

An embodiment of the present disclosure discloses a sanitary napkin having the diagnostic arrangement to detect analytes in menstrual blood. The sanitary napkin includes the top layer to collect menstrual fluid. In an embodiment, sanitary napkin having the diagnostic arrangement includes the middle layer having plurality of parallelly placed vertical columns for a size-based separation of the collected menstrual fluid, wherein the parallelly placed vertical columns have one or more shapes with one or more detection markers coated on a corresponding inner surface to react with one or more analytes in the collected menstrual fluid. In an embodiment, sanitary napkin having the diagnostic arrangement includes the sensor layer having plurality of spots corresponding to the plurality of parallelly placed vertical columns, such that each spot of the plurality of spots senses concentration of the one or more analytes across the corresponding vertical columns.

In an embodiment, the sanitary napkin having the diagnostic arrangement includes the transmitter to wirelessly transmit the sensed concentration to a user device to compare with known normal healthy conditions for detecting and analyzing the one or more analytes in the menstrual blood. In an embodiment, the sanitary napkin having the diagnostic arrangement further includes a bottom layer to sandwich at least one of the middle layer, the sensor layer, and the transmitter with the top layer. The bottom layer of the sanitary napkin sandwiches the middle layer with the sensor layer, and the transmitter with the top layer. Additionally, the top layer has an adhesive to support such applications. Further, the bottom layer is made of a waterproof material to prevent the passing of the menstrual blood.

The features and advantages of the subject matter hereof will become more apparent in light of the following detailed description of selected embodiments, as illustrated in the accompanying FIGUREs. As one of ordinary skill in the art will realize, the subject matter disclosed is capable of modifications in various respects, all without departing from the scope of the subject matter. Accordingly, the drawings and the description are to be regarded as illustrative.

### BRIEF DESCRIPTION OF THE DRAWINGS

The present subject matter will now be described in detail with reference to the drawings, which are provided as illustrative examples of the subject matter to enable those skilled in the art to practice the subject matter. It will be noted that throughout the appended drawings, features are identified by like reference numerals. Notably, the FIGUREs and examples are not meant to limit the scope of the present subject matter to a single embodiment, but other embodiments are possible by way of interchange of some or all of the described or illustrated elements and, further, wherein:

FIG. 1 illustrates an environment of a diagnostic arrangement in a sanitary napkin, according to one or more embodiments of the present disclosure.

FIG. 2 illustrates an exploded view of the sanitary napkin having the diagnostic arrangement, in accordance with an embodiment of the present disclosure.

FIG. 3 illustrates an exemplary coupling of a middle layer of the sanitary napkin with a sensor layer, in accordance with an embodiment of the present disclosure.

FIGS. 4A-4C illustrate various exemplary shapes of vertical channels for various size-based separations of collected menstrual blood, in accordance with an embodiment of the present disclosure.

FIG. 5A-5B illustrate various interfaces of a mobile application, in accordance with an embodiment of the present disclosure.

### DETAILED DESCRIPTION OF THE EMBODIMENTS

The detailed description set forth below in connection with the appended drawings is intended as a description of exemplary embodiments in which the presently disclosed process can be practiced. The term “exemplary” used throughout this description means “serving as an example, instance, or illustration,” and should not necessarily be construed as preferred or advantageous over other embodiments. The detailed description includes specific details for providing a thorough understanding of the presently disclosed method and system. However, it will be apparent to those skilled in the art that the presently disclosed process may be practiced without these specific details.

The terms “connected” or “coupled” and related terms are used in an operational sense and are not necessarily limited to a direct connection or coupling. Thus, for example, two devices may be coupled directly, or via one or more intermediary media or devices. As another example, devices may be coupled in such a way that information can be passed therebetween, while not sharing any physical connection. Based on the disclosure provided herein, one of ordinary skill in the art will appreciate a variety of ways in which connection or coupling exists in accordance with the aforementioned definition.

If the specification states a component or feature “may,” “can,” “could,” or “might” be included or have a characteristic, that particular component or feature is not required to be included or have the characteristic.

As used in the description herein and throughout the claims that follow, the meaning of “a,” “an,” and “the” includes plural reference unless the context dictates otherwise. Also, as used in the description herein, the meaning of “in” includes “in” and “on” unless the context dictates otherwise.

The phrases “in an embodiment,” “according to one embodiment,” and the like generally mean the particular feature, structure, or characteristic following the phrase is included in at least one embodiment of the present disclosure and may be included in more than one embodiment of the present disclosure. Importantly, such phrases do not necessarily refer to the same embodiment.

It will be appreciated by those of ordinary skill in the art that the diagrams, schematics, illustrations, and the like represent conceptual views or processes illustrating systems and methods embodying this invention.

The present disclosure discloses a diagnostic arrangement to detect the presence of multiple vaginal infections which may provide rapid and easy visualisation of results. The diagnostic arrangement may include a top layer, a middle layer, a sensor layer, and a transmitter. The top layer of the diagnostic arrangement may form an upper region, where the menstrual blood having one or more analytes may be collected. The collected menstrual blood having one or more analytes may flow from the top layer to the middle layer of the diagnostic arrangement. Moreover, the middle layer of the diagnostic arrangement may have one or more parallelly arranged vertical columns. Further, the collected menstrual blood from the top layer may undergo size-based separation in the one or more parallelly arranged vertical columns. Additionally, the one or more parallelly arranged vertical columns may have one or more detection markers coated on their corresponding inner surfaces. These one or more detection markers may further react with one or more analytes present in the collected menstrual blood.

In some embodiments, the sensor layer in the diagnostic arrangement may lie below the middle layer. The sensor layer may have one or more spots corresponding to the one or more parallelly placed vertical columns. Each spot of the one or more spots senses the concentration of each analyte of the one or more analytes across all the one or more vertical columns. Additionally, the sensors layer may have an impedance-based sensor on each of the one or more spots, such that the coupling of an antigen with the detection marker may lead to a change in impedance on the corresponding spot. The change in impedance on the corresponding spot may be associated with a concentration of a corresponding analyte binding in the corresponding vertical columns.

In some embodiments, the transmitter in the diagnostic arrangement may transmit the readings of the concentration of each analyte of the one or more analytes, taken by the one or more sensors in the sensor layer to a user devices or applications. The transmitter gives a constant read-out and monitors all the spikes in the readings.

In another embodiment, the diagnostic arrangement may easily be incorporated into sanitary pads, evading the need to purchase separate testing kits. Thus, making it accessible to at-risk individuals who withhold from getting tested without symptoms. Specific analytes of vaginal infectious diseases such as Candidiasis caused by fungi Candida albicans, Gonorrhea causes by Neisseria Gonorrhea, and menstrual associated Toxic-Shock Syndrome (mTSS) caused by Staphylococcus aureus and other analytes of such multiple organisms may be detected. In an embodiment, the proposed diagnostic arrangement in sanitary napkins gives both privacy and control over the next course of action. In many cases, socio-cultural and economic restrictions prevent women from seeking the right treatment or even diagnosing sexual health-related infections. The proposed diagnostic arrangement embedded within sanitary napkins empowers such women to take control of their sexual health and overall well-being. In another embodiment, the diagnostic arrangement may be arranged in other hygiene products like tampons, menstrual cups, etc.

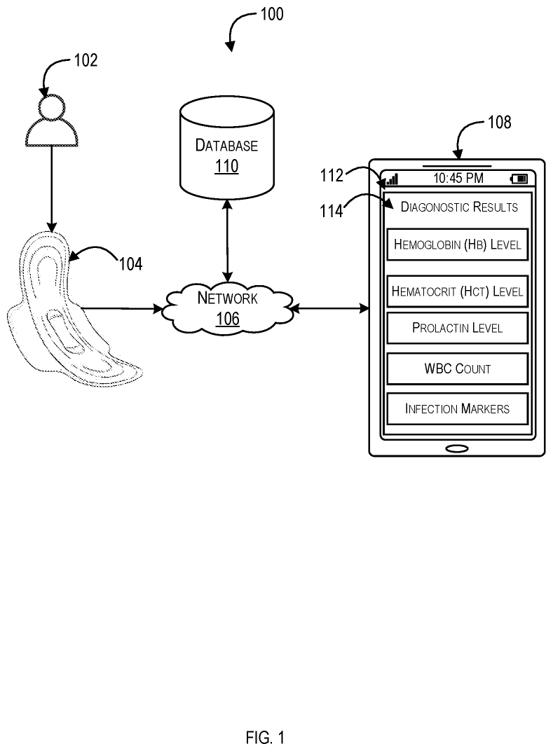

FIG. 1 illustrates an environment 100 of a diagnostic arrangement in a sanitary napkin 104, according to one or more embodiments of the present disclosure. The environment 100 may include a user 102, a sanitary napkin 104, a communication network 106, a user device 108, and a database 110. In an embodiment, the diagnostic arrangement may be arranged in the sanitary napkin 104. In another embodiment, the diagnostic arrangement may be arranged in other sanitary products such as tampons, and menstrual cups. In yet another embodiment, the diagnostic arrangement may be arranged in a specifically made product for detecting like a detection strip. For the sake of brevity, the diagnostic arrangement has been explained as being arranged inside the sanitary napkin 104, but it may be apparent to a person skilled in the art that the other implementation (such as in tampons, menstrual cups, and/or detection strip) may be similarly done without departing from the scope of the present disclosure. Further, the sanitary napkin 104 may communicatively be coupled to a user device via a communication network.

In an embodiment, the sanitary napkin 104 may be associated with the user 102, such that the sanitary napkin 104 may collect menstrual blood from the user 102 in an upper region. In an embodiment, the menstrual blood may have altered pH levels, and contain one or more analytes such as microorganisms, microbial markers, cytokines and inflammatory markers, metabolites, stress biomarkers, vitamins, hemoglobin, iron-based compounds and other minerals, prostaglandins, hormones, blood cells, endometrial tissue, etc. Further, the collected menstrual blood having one or more analytes may undergo a size-based separation in the sanitary napkin 104. Due to such size-based separation, the one or more analytes may be separated into different components such as microbes, white blood cells (WBCs), red blood cells (RBCs), cellular matter, plasma contents, etc. Additionally, the presence of one or more analytes may facilitate the detection of infectious disease conditions including Candidiasis based on fungi Candida albicans, Gonorrhea caused by Neisseria Gonorrhea, and menstrual-associated Toxic-Shock Syndrome (mTSS) caused by Staphylococcus aureus.

In an embodiment, the diagnostic arrangement may have one or more sensors which sense the concentration of the one or more analytes. The readings of the sensed concentration of the detected one or more analytes are taken throughout the sanitary napkin 104 and transmitted, via a transmitter, to the user device 108. In an embodiment, the received readings in the user device 108 are compared with known normal healthy conditions and/or with the historical data of the user 102, stored in the database 110. In an embodiment, the user device 108 may include an interface 112 through which the user 102 may be informed of details regarding the detection and analysis of the analytes in the menstrual blood.

The interface 112 may communicate one or more diagnostic results 114 to the user 102. The communicated one or more diagnostic results may include, but are not limited to, infection markers, microbial load, hemoglobin (HB) level, hematocrit (HCT) level, prolactin levels, WBC Count, etc which may be indicative of infectious diseases, inflammatory conditions, underlying metabolic disorders, vitamin and mineral deficiencies, stress and fertility problems, amongst others. In some embodiment, the interface 112 may also depict qualitative and quantitative changes in the menstrual fluid, preliminary diagnoses, and possible suggestions to improve the existing conditions.

In an embodiment, the interface 112 of the user device 108 may include but is not limited to, a display screen, a microphone, a speaker, and so on. The user device 108 may, without any limitation, include a smartphone, a Personal Assistant Device (PDA), a PC, a tablet, a laptop, a smartwatch, and so on. It may be understood that the transmitter may correspond to any of the existing wireless modules (e.g., Bluetooth module, Infrared module, ZigBee module, or the like) for connecting the sanitary napkin 104 with the user device 108 via the communication network 106 accordingly, the communication network 106 may include, without limitation, a direct interconnection, a Local Area Network (LAN), a Wide Area Network (WAN), a wireless network (e.g., using Wireless Application Protocol), the Internet, and the like.

In some embodiment, the diagnostic arrangement may, without any limitation, be arranged in tampons, menstrual cups, etc. which may be used by women during the menstrual cycle. In another embodiment, the diagnostic arrangement may be exclusively arranged on a strip coated with detection markers specific to the analytes of interest. These strips when in contact with a small portion of menstrual blood induce the detectable changes. The information regarding the detectable changes may further be transmitted wirelessly to the user device 108, via the communication network 106.

In another embodiment, the diagnostic arrangement may detect analytes using various detection processes such as biochemical reactions, dye displacement and visual detection, nanoparticle-based sensing, and so on. If necessary, signal amplification techniques may be employed to enhance the detection process of analytes in the menstrual blood.

In a non-limiting embodiment, the sanitary napkin(s) 104 which are used during the initial days of the menstrual cycle may only be embedded with the diagnostic arrangements. The diagnostic arrangement may be arranged and implemented in the detection of various diseases such as cervical cancer, sexually transmitted diseases, major vitamin deficiencies, and so on. The results from the tests conducted using the diagnostic arrangement may serve as a primer for medical practitioners to better advise their patients and also help patients make informed decisions. In developing economics, these tests may serve as affordable and accessible healthcare. The power of being informed and aware of one's health may help in advancing the overall health of a community.

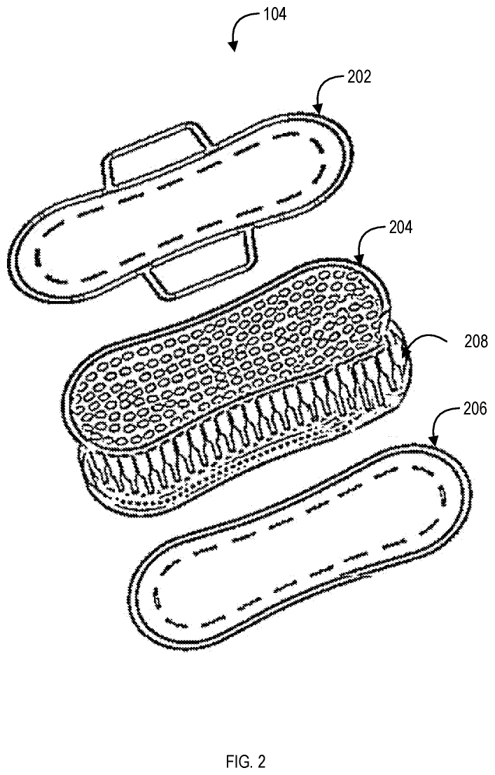

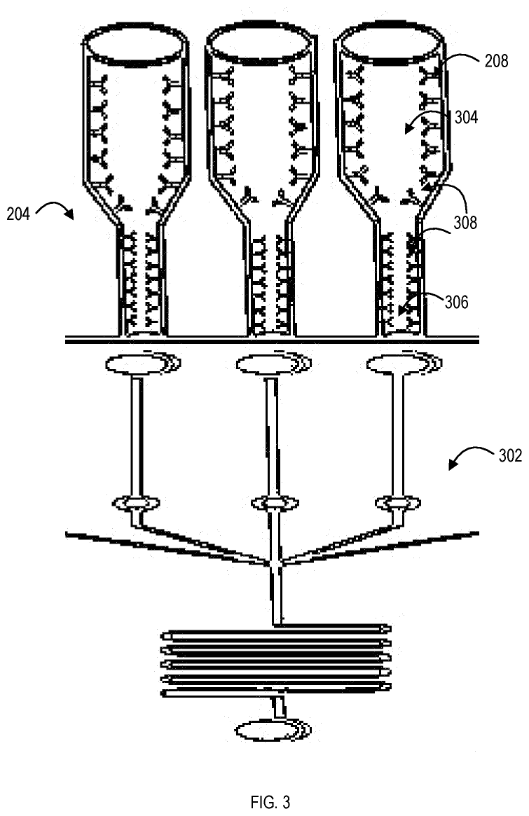

FIG. 2 illustrates an exploded view of the sanitary napkin 104 having the diagnostic arrangement, in accordance with an embodiment of the present disclosure. FIG. 3 illustrates an exemplary coupling of a middle layer 204 with a sensor layer 302, in accordance with an embodiment of the present disclosure. For the sake of brevity, FIGS. 2, and 3 have been explained together.

In an embodiment, the sanitary napkin 104 having a diagnostic arrangement may include a top layer 202, a middle layer 204, a sensor layer 302, a transmitter (not shown in the figure), and a bottom layer 206. The top layer 202 of the diagnostic arrangement may be part of an upper region of the sanitary napkin 104, which may collect the menstrual blood from the user 102. In one scenario, the top layer 202 may be the upper layer of the sanitary napkin 104. In another scenario, the top layer 202 may be attached to the upper layer of the sanitary napkin 104, such that the diagnostic arrangement may be sandwiched between the upper layer of the sanitary napkin 104 and the bottom layer 206 of the sanitary napkin 104. Similarly, the top layer 202 may form part of the upper regions of other products like tampons and/or menstrual cups, as mentioned in the previous paragraphs. In an embodiment, the top layer 202 may be made of a layer of absorbent material. The absorbent material may consist of a porous material that meets the necessary absorbency criteria. Potential materials for the top layer 202 may include but are not limited to, polyester, polyethylene, polypropylene, or their combinations, as well as cotton, viscose, and rayon, among others. Further, the top layer 202 may be blended with super absorbent polymers to boost absorption and maintain a slim profile for the pad. In some embodiments, the top layer 202 may be the contact layer between the diagnostic arrangement and the skin of the user 102. In some other embodiments, the top layer 202 may not be in contact with the user 102 and there may be a necessity to drop, smear, or coat the menstrual blood on the top layer.

In an embodiment, the middle layer 204 may include a plurality of parallelly placed vertical columns 208 for a size-based separation of the collected menstrual fluid. The vertical columns 208 may have one or more detection markers 308 coated on a corresponding inner surface to react with one or more analytes in the collected menstrual fluid. Further, due to the size-based separation, the larger size components such as the endometrial cells, white blood cells (WBCs), red blood cells (RBCs), and other cellular matter are retained at a first region 304 of the vertical column 208, and the smaller size components such as bacteria along with the plasma contents (proteins, nucleic acid) are separated into a second region 306.

In an embodiment, the vertical columns 208 may be made up of a sensitive material and/or may have one or more immobilized detection markers 308 coated on a corresponding inner surface of the vertical columns 208. The one or more immobilized detection markers 308 may be specific to the analytes of interest, from human cells, microbes, or plasma. In an embodiment, the immobilized detection markers 308 may be complementary and/or specific to analytes of interest expressed on the surface of human or microbial cells. The immobilized detection markers 308 may react with one or more analytes present in the collected menstrual blood, which may lead to enabling specific trapping and subsequent detection of these analytes. Such diagnostic arrangement involving the process of separation, coupled with the detection of components specific to the size range, may increase the efficiency of detection by a targeted approach. Further, the immobilized detection markers 308 may include antibodies specific to antigens of interest and/or oligonucleotides bound to complementary nucleic acids.

In an embodiment, the vertical columns 208 may be funnel-shaped as shown in FIG. 3. In the funnel-shaped columns, as shown in FIG. 3, the larger particles and cells may get retained in the upper region 304 of the column, and the smaller plasma contents and bacteria may flow downwards to the narrower region 306 by the principle of size exclusion. Additionally, the deposition of the cells may create a bedding for finer filtration of the menstrual blood.

In an embodiment, the sensor layer 302 may include a plurality of spots corresponding to the vertical columns 208. Additionally, each spot of the plurality of spots senses the concentration of the one or more analytes across the corresponding vertical columns 208 immediately above. In some embodiments, the sensor layer 302 may have an impedance-based sensor on each of the plurality of spots. Moreover, a change in impedance on the corresponding spot may occur due to the coupling of an antigen with the detection marker. The change in impedance on the corresponding spot may be associated with a concentration of a corresponding analyte binding in the corresponding vertical columns. These signals are relayed to one or more sensors in the sensor layer 302. Additionally, the sensors in the sensor layer 302 may, but not limited to, be biosensor, electric, semiconductor, and/or nanomaterial based.

In an embodiment, signal translations in the sensor layer 302 may be done using a function with each parameter represented by the corresponding analyte binding (such as based on the concentration of that analyte existing in the sample). The parameters having dynamic weights may correlate to the impedance/piezoelectric readings obtained specific to that parameter. In this way, the total function with weights of all parameters may be recorded for a particular vertical channel 208 by the corresponding one or more spots on the sensor layer 302. The function corresponding to each spot of the one or more spots may be collected to determine the total concentrations of each analyte across all the one or more vertical columns 208. Further, one or more sensors in the sensor layer 302 may take the readings of the concentration of each analyte across all the one or more vertical columns 208 throughout the middle layer 204 of the sanitary napkin 104.

In an embodiment, the transmitter may transmit the readings of the concentration of each analyte across all the one or more vertical columns 208, taken by the one or more sensors in the sensor layer 302. The transmitter may transmit the readings, via the communication network 106 and may give a constant readout and monitor all spikes in the diagnostic arrangement. In an embodiment, the transmitter may be coupled to an electrical contact placed on a diagnostic arrangement. The electrical contact may be placed to communicate electrically with a corresponding electrical contact of the diagnostic arrangement.

In an embodiment of the sanitary napkin 104 having the diagnostic arrangement, the transmitter may be encased in a waterproof containment arrangement. The waterproof containment arrangement may house a small, appropriate battery (such as a coin cell battery) to power the transmitter. The transmitter may be connected to the battery with safety features like on/off switches and power management circuits, ensuring efficient use of the battery's energy and preventing damage. Safety standards and regulations are crucial when incorporating electronic components and batteries into a sanitary napkin 104 to ensure safety and effectiveness. Further, the transmitter may have active and quiescent modes of operation and may further be provided a timer for timing the duration of a mode of the transmitter. The timer may also provide data related to the duration of a period of wetness of the absorbent layer, as well as the duration of a period of dryness. Additionally, the transmitter may further transmit the data related to moisture levels, menstrual flow, menstrual cycle, and offer smartphone integration for real-time data access and notifications. Additionally, it can provide leak detection, health insights, and user education.

In an embodiment, the transmitter may be arranged to transmit a signal containing the readings of the sensed concentration associated with the transmitter. Further, the user device 108 may receive the signal containing sensed concentration associated with the transmitter. Further, the baseline levels of each of the blood components (cells, plasma proteins, and commensal microbes) at normal healthy conditions may be stored in the database 110, which may be used as a reference for detection and analysis of the concentrations of each analyte in a collected menstrual blood sample. The database 110 may also store the historical data of the user 102 for the analysis of the received information.

In another embodiment, the vertical column 208 may include antibodies or proteins specific to antigens of interest (on the cell surface or suspended in plasma). Further, the immobilized detection markers 308 may include antibodies or proteins designed in such a way that they may bind to molecules expressed on the surface of the blood cells, endothelial cells, or bacteria, in a specific and stable manner. Such proteins may also be able to capture the molecules suspended in the plasma. In another embodiment, the diagnostic arrangement includes nucleic acid sequences which may be tagged to proteins, that may bind complementary nucleic acid sequences present extracellularly. A cell lysis step may be included if necessary. For the aforementioned diagnostic arrangement components like vesicles or beads may be used for additional sequestering and targeting of analytes.

In another embodiment, the bottom layer 206 of the sanitary pad 104 may sandwich the middle layer 204, the sensor layer 302, and or the transmitter with the top layer 202. Additionally, the top layer 202 contains adhesive for such applications. Further, waterproof materials (such as a thin plastic film or a polyethylene material that is impermeable to liquids) are used for the bottom layer 206 so as to prevent the passing of the menstrual blood. The bottom layer 206 may be made of a material that provides comfort and protection to the user 102. In some embodiments, sanitary napkins 104 may incorporate a breathable bottom layer 206. The breathable bottom layer 206 may allow for air circulation, reducing the risk of moisture and discomfort, while still maintaining its leak-proof properties.

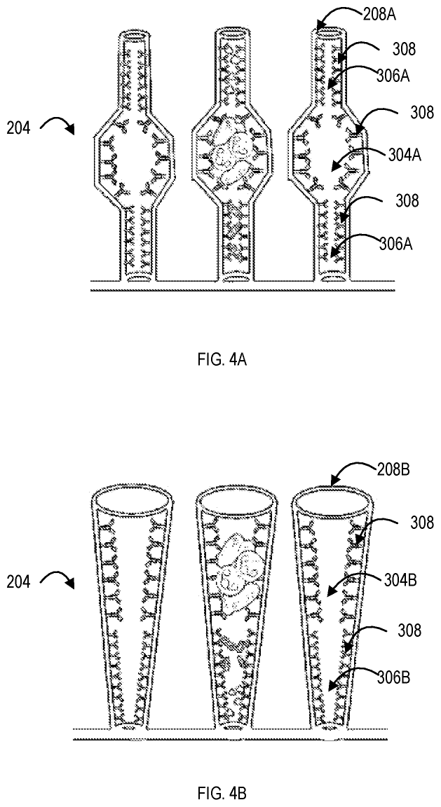

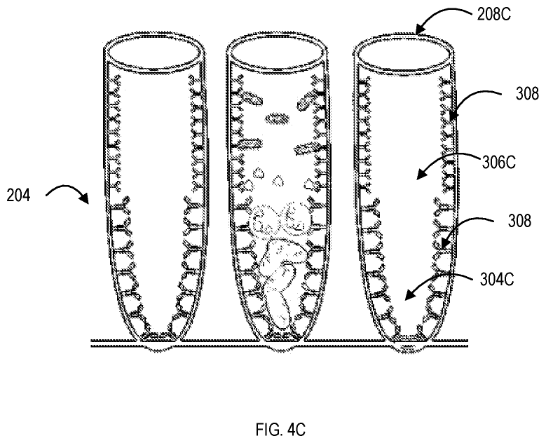

FIGS. 4A-4C illustrate various exemplary shapes of vertical columns 208 for various size-based separations of collected menstrual blood, in accordance with an embodiment of the present disclosure. In an embodiment, the separation of components may be done in various ways based on the requirements of detection, and the detector molecules specific to molecules found in a particular region of separation may be immobilized at that region. Further, to enhance the feasibility of the design and maximize detection efficiency the shape and the size of the vertical columns 208 may be manipulated to suit a specific style of separation. In some embodiments, the vertical column 208 may be a rolling pin column as shown by 208A, a Conical tapering column as shown by 208B, and/or a cylindrical column as shown by 208C. In the rolling pin-shaped vertical column, as shown by 208A, due to sedimentation and size exclusion the larger contents of the menstrual blood may get held in the wide, middle first region 304A of the channel, while the smaller contents may be located at the top and the bottom second regions 306B respectively. Furthermore, in the Conical tapering-shaped column, as shown by 208B, the larger particles and cells may be restricted to the first wider portion 304B of the cone, and the smaller particles may easily flow through the gaps and settle towards the second bottom 306B, similar to the size exclusion principle. Whereas, in the cylindrical column, as shown by 208C, the components may be separated based on sedimentation, where a uniform diameter of the channel may enable the larger cells and particles to settle at the first bottom 304C by virtue of their density while the smaller particles in the menstrual blood may form second layers above 306C the densest layer at the bottom, creating a density-based sedimentation gradient.

In another embodiment, other possible designs may include but are not limited to, columns and/or channels with multiple constrictions, charge-based separation, or even the presence of multiple strata within the detection layer for the different components. In another embodiment, these columns/channels may also function as packed columns, packed with a gel-like matrix which may aid in the finer separation of the particles based on their size. In some embodiments, the components in the column could also be charged, to optimize separation. Furthermore, several permutations of the aforementioned designs may be used based on the separation efficiency and practical implications of the particular design. The cross-sectional widths of the columns may be customized according to the size and expected concentration of each component of the menstrual blood, as well as their behavior during deposition and/or collection.

In some embodiments, the vertical columns 208 with different shapes and sizes including the rolling pin column as shown by 208A, a Conical tapering column as shown by 208B, and/or a cylindrical column as shown by 208C may be made up of a sensitive material and/or immobilized with detection markers 308. Similarly, the inner surfaces of all the vertical columns 208 including the rolling pin column as shown by 208A, a Conical tapering column as shown by 208B, and/or a cylindrical column as shown by 208C may contain one or more immobilized detection markers 308 coated on a corresponding inner surface.

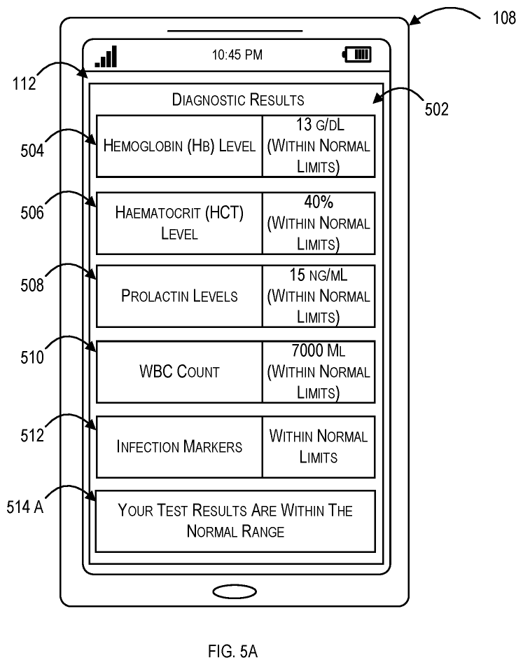

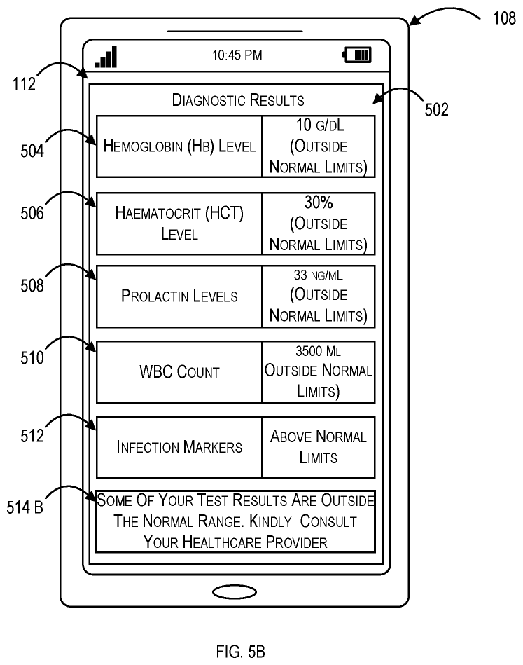

FIG. 5A-5B illustrate various interfaces 112 of a mobile application, in accordance with an embodiment of the present disclosure. In some embodiments, the interface 112 of the user device 108 may communicate the diagnostic result 502 to the user 102. The one or more diagnostic results 502 may include, but not limited to, hemoglobin (Hb) Level 504, Hematocrit (HCT) Level 506, Prolactin Levels 508, WBC Count 510, Infection Markers 512, etc.

In some embodiments, when the communicated results are within the normal limits, the user 102 may be informed of their well-being. For example, in FIG. 5A as the test results are normal the information 514A communicated to the user 102 may be ‘Your test results are within the normal range’. In another embodiment, when one or more test results are outside the normal limits, the user 102 may be informed of their ill health and the information may contain some suggestion. For example, in FIG. 5B as the test results are outside or above normal limits the information 514B may be communicated to the user 102 may be ‘Some of your test results are outside the normal range. Kindly consult your healthcare provider’.

In a non-limiting embodiment, the proposed diagnostic arrangement in the sanitary napkin 104 may provide results as soon as the pathogen numbers cross the safe limit. Integration of the diagnostic arrangement into the sanitary napkin 104 may increase its accessibility when compared to laboratory tests and expensive diagnostic kits. The diagnostic arrangements in the sanitary napkin 104 may detect specific infections and may be self-sustaining, cheap, all-in-one diagnostic kits in addition to functioning as a normal sanitary napkin 104. The proposed sanitary napkins 104, with the embedded diagnostic arrangement, may be compact, discrete, and private, and only the user 102 would be able to access the test results. Also, as the proposed diagnostic arrangement seamlessly integrates into existing menstrual hygiene products like sanitary pads other external detection instruments may not be needed for regular check-ups.

In an implementation, the diagnostic arrangement in sanitary health products may enable the early detection and prevention of diseases including but not limited to Candidiasis caused by the fungi Candida albicans, Gonorrhea caused by Neisseria Gonorrhea or disorders such as Menstrual associated Toxic-shock syndrome (mTSS) caused by Staphylococcus aureus. Immobilizing antibodies complementary or specific to antigens expressed on the surface may enable specific trapping and subsequent detection of these pathogens. For example, Coagulase, an enzyme secreted by Staphylococcus aureus may bind to Prothrombin present in non-menstrual human blood. Antibodies may be designed with binding pockets similar to prothrombin and immobilized on the diagnostic arrangement and upon coagulase-binding, a signal may be observed, indicating the presence of the bacteria.

Menstrual-associated infections are known to peak towards the end of the menstrual cycle, mainly due to unsafe menstrual hygiene practices. Pre-existing methodologies of diagnosis are time-consuming, and these diseases often go unnoticed until the arrival of late-onset symptoms. Early qualitative and quantitative detection of bacterial or fungal units in the vaginal tract may allow the identification of infectious disease conditions. In this way, venereal diseases, symptoms of poor hygiene, or even complications due to lack of timely removal and change of menstrual sanitary products may be detected as a preliminary diagnosis.

In another implementation, underlying chronic and/or systemic conditions may also be detected which may have remained hidden due to the presence of various components such as hormones, cytokines, and/or other inflammation-like fluctuations. The various underlying chronic and/or systemic conditions may be identified when there is a deviation from the normal levels. This may include but is not limited to conditions such as Polycistronic Ovary Syndrome/Disorder (PCOS/D) and cancer. An increase in cytokine levels is synonymous with cancer-like conditions and the vaginal discharge could provide great insights into the levels of various cytokines secreted. Hormones are secreted in abundance throughout a menstrual cycle and the hormone profiles vary from each individual. Elevated levels of testosterone are usually detected in individuals who have or are prone to PCOD, and they may also be determined by the sensors in the disclosed sanitary health product.

In an implementation, the detection of certain blood biomarkers may indicate potential lifestyle disorders and may even suggest ways for customers or users to monitor themselves (stress levels, anxiety, sleep cycles) using a mobile application. Based on a few inputs from the customer or users, the disclosed sanitary health product could be configured to moderate the levels for the user and customize the feedback to suit their needs better. Levels of lifestyle hormones such as cortisol, serotonin, dopamine, and other endorphins may be sensed through menstrual discharge. Levels of essential nutrients in the blood, including vitamins, minerals, and other metabolites may be checked to detect abnormal fluctuations, or underlying disorders such as deficiencies, diabetes mellitus, and infertility, amongst others. During the course of a period, the readings may provide insight into the development of any menstrual-associated disorders.

In another implementation, the baseline may adapt to a person's body temperatures and other lifestyle norms, providing more accurate period predictions than date-based period trackers. This may also help keep track of the person's fertility cycle and may aid in planned pregnancies, reducing dependency on artificial methods by those who are able. Most of these menstrual-associated disorders arise from a lack of awareness or ignorance of menstrual hygiene practices. Regularly changing disposable menstrual products is the key to maintaining proper vaginal health. The menstrual discharge, once collected in a sanitary product, begins to fester an environment greatly suited to microbial growth. The signals from the sanitary health product may thus indicate when the microbial load has reached an unsafe threshold on the sanitary health product and prompt the user to change their sanitary napkin or tampon, thereby promoting menstrual hygiene.

While embodiments of the present disclosure have been illustrated and described, it will be clear that the disclosure is not limited to these embodiments only. Numerous modifications, changes, variations, substitutions, and equivalents will be apparent to those skilled in the art, without departing from the spirit and scope of the disclosure, as described in the claims. Thus, it will be appreciated by those of ordinary skill in the art that the diagrams, schematics, illustrations, and the like represent conceptual views or processes illustrating systems and methods embodying this disclosure.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

While the foregoing describes various embodiments of the disclosure, other and further embodiments of the disclosure may be devised without departing from the basic scope thereof. The scope of the disclosure is determined by the claims that follow. The disclosure is not limited to the described embodiments, versions, or examples, which are included to enable a person having ordinary skill in the art to make and use the disclosure when combined with information and knowledge available to the person having ordinary skill in the art.

What is claimed:

1. A diagnostic arrangement to detect analytes in menstrual blood, the diagnostic arrangement comprising:
a top layer to collect the menstrual fluid;
a middle layer having a plurality of parallelly placed vertical columns for a size-based separation of the collected menstrual fluid, wherein the parallelly placed vertical columns having one or more shapes with one or more detection markers coated on a corresponding inner surface to react with one or more analytes in the collected menstrual fluid;
a sensor layer having a plurality of spots corresponding to the plurality of parallelly placed vertical columns, such that each spot of the plurality of spots senses concentration of one of the one or more analytes across the corresponding vertical columns; and
a transmitter to wirelessly transmit the sensed concentration to a user device, such that the user device compares the sensed concentration with known normal healthy conditions for detecting and analysing the one or more analytes in the menstrual blood.

2. The diagnostic arrangement of claim 1, wherein the one or more shapes include at least one of: a funnel shape, a rolling pin shape, a conical tapering shape, and a cylindrical shape.

3. The diagnostic arrangement of claim 1, wherein the one or more detection markers include at least one of: antibodies specific to antigens of interest and oligonucleotides bound to complementary nucleic acids.

4. The diagnostic arrangement of claim 1, wherein the size-based separation forms a first region of larger size components and a second region of smaller size components.

5. The diagnostic arrangement of claim 4, wherein the larger size components include cellular matter having at least one of: endometrial cells, White Blood Cells (WBCs), and Red Blood Cells (RBCs).

6. The diagnostic arrangement of claim 4, wherein the smaller size components include bacteria along with plasma content having at least one of: proteins and nucleic acid.

7. The diagnostic arrangement of claim 1, wherein the sensors layer has an impedance-based sensors on each of plurality of spots, such that the coupling of an antigen with the detection marker leads to change in impedance on the corresponding spot.

8. The diagnostic arrangement of claim 1, wherein the change in impedance on the corresponding spot is associated with a concentration of a corresponding analyte binding in the corresponding vertical columns.

9. The diagnostic arrangement of claim 1, wherein the analytes facilitate detection of infectious disease conditions including at least one of: candidiasis based by fungi Candida albicans, gonorrhea caused by Neisseria gonorrhea, and menstrual associated Toxis-Shock Syndrome (mTSS) caused by Staphylococcus aureus.

10. The diagnostic arrangement of claim 1, wherein the top layer, the middle layer, the sensor layer, and the transmitter are comprised in at least one of: a sanitary napkin and a tampon.

11. A sanitary napkin having diagnostic arrangement to detect analytes in menstrual blood, the sanitary napkin comprising:
a top layer to collect the menstrual fluid;
a middle layer having a plurality of parallelly placed vertical columns for a size-based separation of the collected menstrual fluid, wherein the parallelly placed vertical columns having one or more shapes with one or more detection markers coated on a corresponding inner surface to react with one or more analytes in the collected menstrual fluid;
a sensor layer having a plurality of spots corresponding to the plurality of parallelly placed vertical columns, such that each spot of the plurality of spots senses concentration of one of the one or more analytes across the corresponding vertical columns;
a transmitter to wirelessly transmit the sensed concentration to a user device, such that the user device compares the sensed concentration with known normal healthy conditions for detecting and analysing the one or more analytes in the menstrual blood, and
a bottom layer to sandwich at least one of: the middle layer, the sensor layer, and the transmitter with the top layer.

12. The sanitary napkin of claim 11, wherein the one or more shapes include at least one of: a funnel shape, a rolling pin shape, a conical tapering shape, and a cylindrical shape.

13. The sanitary napkin of claim 11, wherein the one or more detection markers include at least one of: antibodies specific to antigens of interest and oligonucleotides bound to complementary nucleic acids.

14. The sanitary napkin of claim 11, wherein the size-based separation forms a first region of larger size components and a second region of smaller size components.

15. The sanitary napkin of claim 14, wherein the larger size components include cellular matter having at least one of: endometrial cells, White Blood Cells (WBCs), and Red Blood Cells (RBCs).

16. The sanitary napkin of claim 14, wherein the smaller size components include bacteria along with plasma content having at least one of: proteins and nucleic acid.

17. The sanitary napkin of claim 11, wherein the sensors layer has an impedance-based sensor on each of plurality of spots, such that the coupling of an antigen with the detection marker leads to change in impedance on the corresponding spot.

18. The sanitary napkin of claim 11, wherein the change in impedance on the corresponding spot is associated with a concentration of a corresponding analyte binding in the corresponding vertical columns.

19. The sanitary napkin of claim 11, wherein the analyses facilitate detection of infectious disease conditions including at least one of: candidiasis based by fungi Candida albicans, gonorrhea caused by Neisseria gonorrhea, and menstrual associated Toxic-Shock Syndrome (mTSS) caused by Staphylococcus aureus.

20. The sanitary napkin of claim 11, wherein the top layer having an adhesive for application, and the bottom layer made of a waterproof material to prevent the passing of the menstrual blood.
